(19) United States
(12) Patent Application Publication
(10) Pub. No.: US 2009/0117231 A1
(43) Pub. Date: May 7, 2009

(54) Soy Protein Composition and Beverage Products Containing the Soy Protein Composition

(75) Inventors: Michael Murray, House Springs, MO (US); Jimbin Mai, St. Louis, MO (US)

Correspondence Address: Solae, LLC, 4300 Duncan Avenue, Legal Department E4, St. Louis, MO, 63110, US

(73) Assignee: Solae, LLC, St. Louis, MO (US)

(21) Appl. No.: 12/265,309

(22) Filed: Nov. 5, 2008

Related U.S. Application Data
(60) Provisional application No. 60/985,750, filed on Nov. 6, 2007.

(51) Int. Cl. A23L 1/304; C07K 14/415; C07K 1/30; A23L 2/02; A23C 9/152

(52) U.S. Cl. 426/74; 530/378; 530/421; 426/599; 426/580

(57) ABSTRACT

In one aspect, the present invention is directed to isolated soy protein compositions and to the preparation of the isolated soy protein compositions. In another aspect, the present invention is directed to beverage compositions that contain the isolated soy protein compositions and to the preparation of the beverage compositions that contain the isolated soy protein compositions. The beverage is selected from the group consisting of an aqueous acidic liquid or a liquid milk product. The beverage may be prepared and consumed as a cold beverage or a hot beverage or at any temperature of comfort between a cold temperature and a hot temperature. Cold beverages include juices, energy drinks, iced teas, iced coffees, etc. Hot beverages include lattes, mochas, espressos, cappuccinos and teas.

### FIELD OF THE INVENTION

The present invention relates to isolated soy protein compositions and to a process for making said isolated soy protein compositions. The present invention further relates to beverage compositions comprising the isolated soy protein compositions and to methods of making the beverage compositions containing the isolated soy protein compositions.

### BACKGROUND OF THE INVENTION

Juices and other acidic juice-like beverages are popular commercial products. Consumer demand for nutritional healthy beverages has led to the development of nutritional juice or juice-like beverages containing protein. The protein provides nutrition in addition to the nutrients provided by the components of the beverage. Recently it has been discovered that certain proteins have specific health benefits beyond providing nutrition. For example, soy protein has been recognized by the United States Food and Drug Administration as being effective to lower blood cholesterol concentrations in conjunction with a healthy diet. In response, there has been a growing consumer demand for acidic juice-like beverages containing proteins that provide such specific health benefits.

Nutrition is one of the cornerstones of health, well-being, and the prevention of numerous chronic diseases. Nutritional products play an important role in these areas and attempts to provide readily available and convenient nutritional products to the general public has been a major focus in recent years. To remain healthy one must receive essential nutrients which are indispensable to human nutrition. Essential nutrients include both macronutrients, such as fats, carbohydrates and proteins, and micronutrients, such as vitamins and minerals (including trace elements and electrolytes).

Milk products constitute a significant portion of the overall diet or calorie consumption of human beings. As such, milk products play a major role in maintaining the health of the public. Nutritionally optimal milk products will have a positive effect on the nutrition and the health of the public. Concentration of macronutrients in any given milk product will often depend on the nature of the product and the desirable profile developed by the manufacturer.

For example, bovine milk contains 87% water, 3% protein, 0.65% whey, 4.5% to 5.0% lactose, 3% to 4% milk fat, 0.3% to 0.7% mineral salt plus a variety of water and fat soluble vitamins, lactic and citric acids, urea, free amino acids and polypeptides. One or more of these components may be separated from milk to produce various compositions. For example, in the manufacture of cottage cheese or casein, milk fat is first separated centrifugally (as cream) and the casein fraction of the milk is then precipitated at its isoelectric point by the addition of acid. The remainder of the original milk, containing all of the other components listed above, is called whey i.e., milk, from which the casein and a majority of the milk fat has been removed is referred to as whey.

Plant materials, such as soybeans, are processed to produce a wide variety of food products. Recently, consumer demand for low- or reduced-fat, high-protein plant-derived products has increased dramatically.

Protein isolates that are derived from vegetable protein sources, such as soybeans have contributed substantially to the economic importance of these materials as a crop. Soy protein isolates in particular have proven to be a useful nutritional supplement in a variety of foods and beverages. A protein isolate can be generally characterized as a product resulting from the extraction, subsequent concentration, and purification of proteinaceous material from a proteinaceous source such as a vegetable protein material. Typically, the protein isolate on a moisture free basis will have a protein content which will range between about 90% and 98% by weight.

The usefulness of soy protein isolates in the formation of foodstuffs such as beverages has for the most part been accomplished by the production of modified or enzymatically hydrolyzed isolates or the addition of materials such as surfactants to promote the dispersibility or suspendibility of the isolate in the particular type of aqueous medium that is used in preparation of the beverage.

Many food and beverage products include protein supplements derived from vegetable materials such as soybeans, beans, peas, other legumes, and oilseeds such as rapeseed. Vegetable protein materials, particularly soy, are used in beverage applications.

### SUMMARY OF THE INVENTION

In one aspect, the present invention is directed to isolated soy protein compositions and to the preparation of the isolated soy protein compositions. In another aspect, the present invention is directed to beverage compositions that contain the isolated soy protein compositions and to the preparation of the beverage compositions that contain the isolated soy protein compositions. The beverage is selected from the group consisting of an aqueous acidic liquid or a liquid milk product. The beverage may be prepared and consumed as a cold beverage or a hot beverage or at any temperature of comfort between a cold temperature and a hot temperature. Cold beverages include juices, energy drinks, iced teas, iced coffees, etc. Hot beverages include lattes, mochas, espressos, cappuccinos and teas.

The isolated soy protein composition is a soy protein material that contains from about 1.0 wt. % up to about 10 wt. % of a metal salt.

### DETAILED DESCRIPTION OF THE INVENTION

The present invention provides an instant or ready-to-drink beverage of a soy protein composition further containing a juice, a milk product or plain water. More specifically, the present invention provides a high quality instant or ready-to-drink beverage of a soy protein composition with a juice or juice solids, milk or milk solids or water or water fortified with vitamins and minerals. The ready-to-drink beverage may also contain, in addition to water, a fat component and/or a beverage base such as coffee extract solids, spray dried coffee, or tea solids or extract or cocoa, and wherein the beverage results in a product with a smooth mouth feel and with a minimum of settling or non-dispersibility of the soy protein product.

The present invention also provides for a dry blended beverage of a soy protein composition. The soy protein composition for a dry blended beverage contains in addition to a soy protein component, combinations of flavoring agents, coloring agents, defoamers, sweeteners, and nutrients. A dry blended beverage is a powder that is added to a liquid which is then mixed and consumed. In contrast, a ready to drink beverage is a beverage already prepared, all that is required is to open the ready to drink container and then consume.

### Juice

In one embodiment of the invention, the aqueous acidic liquid is a fruit or vegetable juice. Preferred fruit and vegetable juices include apple juice, grape juice, orange juice, carrot juice, lemon juice, lime juice, grapefruit juice, pineapple juice, cranberry juice, peach juice, pear juice, celery juice, cherry juice, tomato juice, passion fruit juice, and juice blends. The fruit and vegetable juices that may be employed in the invention may be obtained from a selected fruit or vegetable by crushing, squeezing, or pressing the fruit or vegetable. The resulting juice may be filtered, strained or passed through a sieve, resin bed, clay or diatomaceous earth bed or filters to remove juice pulp and other materials that are insoluble in the juice, as desired.

In another embodiment of the invention, the aqueous acidic liquid may be formed of water and an acidulent, and, if desired, flavoring, coloring agents, nutrients, and sweeteners. Citric acid, phosphoric acid, lactic acid, ascorbic acid, and other edible acids may be used as acidulents to adjust the pH of the water to the desired pH. The type of acid used as the acidulent should be selected to provide the desired organoleptic properties to the liquid since the type of acid used may significantly affect these properties. For example, lactic acid tends to impart a fermented character to the liquid, citric acid provides a sharp character, and phosphoric acid provides a milder flavor. A blend of acids may be used as the acidulent in order to obtain the desired flavor profile.

Flavoring, coloring agents, nutrients, defoamers, and sweeteners are preferably included with the water and acidulent to provide the liquid with a desired flavor, color, and nutritional profile. The flavoring agents that may be used to flavor the liquid include both natural and artificial flavors. In a preferred embodiment, the flavoring agent is a concentrated fruit or vegetable juice added to the liquid in an amount effective to provide a juice beverage. Coloring agents that may be used to color the liquid include commercially available natural and artificial colorants for aqueous liquids. Nutrients such as vitamins and minerals may also be added to the liquid. Sweeteners may be added to provide desired flavor to the liquid. Preferred sweeteners are carbohydrates such as sucrose and fructose. A particularly preferred sweetening agent is high fructose corn syrup. Defoamers may also be added to the liquid to inhibit protein induced foaming. The flavoring and sweetening agents are included in the liquid in an amount effective to provide the desired taste to the liquid, the nutrients are included to provide the liquid with a desired nutritional profile, and the coloring agents are included in the liquid in an amount effective to provide the desired color to the liquid.

### Milk

Milk is a mixture of proteins of casein and whey proteins wherein the milk is obtained the milking of females of a mammalian species of animals selected from the group consisting of cow, sheep, goat, water buffalo, camel, and mixtures thereof. Generally, however, cows' milk is the preferred dairy liquid used in the practice of the invention.

Casein is a phosphoprotein that exists in milk in the form of rather large colloidal particles containing the protein and also considerable quantities of calcium and phosphate and a little magnesium and citrate. These particles can be separated from milk by high-speed centrifugation, leaving the whey proteins and dissolved constituents in solution. They are commonly referred to as “calcium phosphocaseinate” or “calcium caseinate-phosphate.” Casein can be removed from milk in a number of ways besides high-speed centrifugation. The fundamental definition of casein is operational—it is defined as that protein precipitated from milk by acidification to pH 4.6 to 4.7. The calcium and phosphate associated with casein in the original particles progressively dissolved as the pH is lowered until, at the isoelectric point of pH 4.6 to 4.7, the casein is free of bound salts. A second important means of removing casein from milk is by rennet coagulation. The enzyme rennin has the ability to slightly change casein so that it coagulates in the presence of divalent cations such as calcium. This process is used in preparation of cheese curd. It involves the coagulation of the calcium caseinatephosphate particles as such because the pH does not drop and colloidal calcium and phosphate are not dissolved. Thus, the product prepared by rennet coagulation has high ash content as compared with acid-precipitated casein. Since they are stabilized by charge, the caseinate particles are extremely sensitive to changes in ionic environment. They readily aggregate with increase in concentration of these ions. Since their equilibrium dispersion in milk is rather precarious, minor changes in salt balance and pH easily upset this equilibrium and tend to destabilize and precipitate the casein particles. Whey proteins are composed of different fractions mainly lactalbumin and lactoglobulin. Milk contains approximately about 2.5% casein and about 0.6% whey proteins.

Milk protein is preferably supplied by a highly enriched preparation of milk protein which contains less than about 30 percent of other milk components. Milk protein used in the current invention may be a milk protein concentrate or a milk protein isolate, for example. Milk protein concentrates, milk protein isolates, and other appropriate sources of milk proteins are well-known in the food sciences and are available commercially. Examples include ALAPRO 4700 and TMP 1220 (New Zealand Milk Products, Santa Rosa, Calif.) and Nutrilac CH7813 (Arla Foods Ingredients, Videbaek, Denmark).

Milk obtained by milking one or more cows is referred to as “cow's milk”. Cow's milk, whose composition has not been adjusted, is referred to herein as “whole Milk”. It is comprised of casein, whey proteins, lactose, minerals, butterfat (milkfat), and water.

The composition of “cow's milk” can be adjusted by the removal of a portion of or all of any of the constituents of whole milk, or by adding thereto additional amounts of such constituents. The term “whole milk” is applied the cow's milk that contains at least 3.25% fat. The term “skim milk” is applied to cow's milk from which sufficient milkfat has been removed to reduce its milkfat content to less than 0.5 percent by weight, and typically to less than 0.1%. The term “low fat milk” (or “part-skim milk”) is applied to cow's milk from which sufficient milkfat has been removed to reduce its milkfat content to the range from about 0.5 to about 2.0 percent by weight, with the 1% and 2% varieties widely marketed.

The additional constituents are generally added to cow's milk in the form of cream, concentrated milk, dry whole milk, skim milk, or nonfat dry milk. “Cream” means the liquid, separated from cow's milk, having a high butterfat content, generally from about 18 to 36 percent by weight. “Concentrated milk” is the liquid obtained by partial removal of water from whole milk. Generally, the milkfat (butterfat) content of concentrated milk is not less than 7.5 weight percent and the milk solids content is not less than 25.5 weight percent. “Dry whole milk” is whole milk having a reduced amount of water. It generally contains not more than five percent by weight of moisture on a milk solids not fat basis. “Nonfat dry milk” is the product obtained by the removal of water only from skim milk. Generally, its water content is not more than five weight percent and its milkfat content is not more than 1.5 weight percent.

Thus, the term “cow's milk” includes, among others, whole milk, low fat milk, (part-skim milk), skim milk, reconstituted milk, recombined milk, and whole milk whose content has been adjusted. As such, in this invention, milk is selected from the group consisting of whole milk, skim milk, part-skim milk, reconstituted milk products and recombined milk products.

The beverage may further comprise a thickening agent. Thickening agents can include maltodextrins, pectins, natural and synthetic gums and natural or chemically modified starches or mixtures thereof. The thickening agent should be easily water-soluble. Preferably, a combination of maltodextrin and pectin are used. Most preferably, maltodextrin with a dextrose equivalent of approximately 9-12% is used in combination with a high ester citrus pectin.

The beverage may comprise a sweetener or a combination of sweeteners. The sweetener(s) may be any sweetener(s) normally used in food processing, either natural or artificial, for example sugar alcohols and sugars such as sucrose, fructose, dextrose, maltose, lactose, high fructose corn syrup solids, sorbitol, or mixtures thereof. The sweetener(s) may comprise any suitable synthetic or natural sweetener, which may be a higher intensity sweetener and used in combination with the sugar or sugar alcohol. Examples of such sweeteners include, for example, sucralose, acesulfame potassium, and mixtures thereof. The sweetener may further comprise a mixture of natural or synthetic sweeteners, such as a sugar or sugar alcohol, used in combination with, for example, a high intensity sweetener. Any mixture or combination of natural or artificial sweeteners may be used. Other sweeteners normally used in food or beverage processing can be used if desired. A preferred sweetener is sucrose or a mixture of sucrose with sucralose and acesulfame potassium. Typically, the sweetener(s) will be present in an amount or amounts to provide a desired sweetness and a typical range is from about 0.5% to about 10% by weight of the total composition. One useful sweetener combination comprises from about 0.5% to about 0.75% by weight sucrose, from about 0.04% to about 0.05% by weight sucralose and from about 0.005% to about 0.10% by weight acesulfame potassium, all by weight of the total beverage composition. In accordance with another aspect of the invention, the sweetener may comprise from about 2.9% to about 10% by weight sucrose, by weight of the total beverage composition.

The beverage may further comprise fortifying vitamins or minerals. Any vitamin or mineral normally used in food processing can be used, such as but not limited to, ascorbic acid, biotin, folic acid, niacinamide, riboflavoid, tocopherol acetate, cyanocobalamin, phytonadione, chromium, copper, iron, magnesium, manganese, molybdenum, iodine, selenium, zinc, or phosphorus or mixtures thereof. The most preferred vitamin used in the beverage is ascorbic acid. Any other vitamin or mineral normally used in food or beverage processing may be used if desired.

The beverage may further comprise a flavor component, either natural or artificial, as may be desired, such as, for example and not as a limitation, almond, amaretto, anise, apple, brandy, caramel, cappuccino, cider, cinnamon, cherry, chocolate, chocolate mint, cocoa, coffee, crème de menthe, french vanilla, grape, hazelnut, irish cream, lemon, macadamia nut, mocha, orange, peach, peppermint, pistachio, strawberry, vanilla, wintergreen or mixtures thereof. Any other flavor normally used within the food or beverage processing industry may be utilized. Preferred flavors for the beverage include almond, amaretto, caramel, cappuccino, cider, cinnamon, chocolate, chocolate mint, cocoa, coffee, crème de menthe, hazelnut, mocha, peppermint, vanilla or mixtures thereof. The most preferred flavors include cocoa, vanilla, caramel and chocolate mint. Typically, the flavor or flavors are present in an amount of from about 0.1% to about 0.75% by weight of the total beverage composition.

Soy protein materials useful in the suspendible composition of the present invention include soy protein isolates from which hulls, hypocotyls, and insoluble carbohydrates and polysaccharides have been removed. The preparation of soy protein materials is described in U.S. Pat. No. 5,858,449, which is incorporated herein by reference. The soy protein product is produced by mixing high sucrose, low stachyose soy flakes, water and caustic in an agitated, heated tank for protein and sugar extraction. Once thoroughly heated and agitated the mixture is fed to a first centrifugal separator. The liquor is sent to a holding tank, and the centrate is mixed with water in a separate tank where it is heated and agitated. The mixed centrate is fed to a second centrifugal separator, and the liquor from the second separator is combined with the liquor from the first separator. The combined liquors are pasteurized in a heat exchanger, evaporated to a sufficient solids composition for economical spray drying, and spray dried to provide the novel soy protein product.

The most preferred soy protein material useful in the suspendible protein composition of the present invention is a soy protein isolate. A soy protein isolate is derived by processing whole soybeans to separate soy protein from other soy materials such as the hull, germ, fats and oils, and carbohydrates. As used herein, the term “soy protein isolate” refers to a soy protein material containing at least 90% soy protein by weight on a moisture-free basis.

To form the inventive soy protein isolate, whole soybeans are cracked, dehulled, degermed, and defatted according to conventional procedures in the art to form soy flakes, soy flour, soy grits, or soy meal, which are commercially available starting materials for the production of soy protein isolates. The soy flakes, soy flour, soy grits, or soy meal is/are extracted with an aqueous alkaline solution, typically a dilute aqueous sodium hydroxide solution having a pH of from 7.5 to 11.0, to extract protein from insolubles, primarily insoluble carbohydrates. An aqueous alkaline extract containing the protein is subsequently separated from the insolubles. At this point, a metal salt may be added, followed by treating the extract with an acid to lower the pH of the extract to around the isoelectric point of the soy protein, preferably to a pH of from 4.0 to 5.0, and most preferably to a pH of from 4.4 to 4.6. The soy protein precipitates from the acidified extract. This precipitated soy protein is typically referred to as a soy protein curd. The remaining liquid portion is referred to as the whey. If a metal salt was not added to the above extract, the metal salt may be added after curd formation. The curd containing the metal salt is separated from the extract. The separated protein may be washed with water to remove residual soluble carbohydrates and ash from the protein material. The separated protein is then dried using conventional drying means to form the inventive soy protein isolate.

Modified soy protein materials may also be used as the protein material of the suspendible composition. Soy protein materials may be modified by several techniques known in the art such as protein hydrolysis, by enzyme or by acid treatment, and deamidation by enzymatic treatment.

The metal of the metal salt that is added to the soy protein curd is selected from the group consisting of sodium, potassium, and combinations thereof. The anion portion of the metal salt is selected from the group consisting of a citrate, a phosphate, a hexametaphosphate, and combinations thereof. The metal citrate is selected from the group consisting of sodium citrate, potassium citrate, and mixtures thereof. The metal phosphate is selected from the group consisting of sodium phosphate, potassium phosphate, and mixtures thereof. The metal hexametaphosphate is selected from the group consisting of sodium hexametaphosphate, potassium hexametaphosphate, and mixtures thereof.

In an alternative embodiment for the inclusion of the metal salt, the metal salt may be formed in situ by the reaction of a metal hydroxide selected from the group consisting of sodium hydroxide, potassium hydroxide, and mixtures thereof with an acid selected from the group consisting of citric acid, phosphoric acid, hemametaphosphoric acid, and mixtures thereof.

The metal salt is added either before or after curd formation or both before and after curd formation in an amount such that the spray dried soy protein isolate contains from about 1 wt. % up to about 10 wt. %, preferably from about 1 wt. % up to about 6 wt. %, and most preferably from about 1 wt. % up to about 3 wt. %.

It is surprising that the addition of the above metal salts during processing of the soy protein composition leads to an improvement in the hydration of the soy protein composition when the soy protein composition is added to a liquid.

The invention of the metal salt containing soy protein isolate having been generally described above, it may be better understood by reference to the examples described below. The following examples represent specific but non-limiting embodiments of the formation of the metal salt containing soy protein isolate of the present invention

### Example 1

Added to a mixture of soy protein curd and whey protein, wherein there are 70 parts of the protein curd containing 10% soy protein are 0.1 parts of anhydrous citric acid and 0.06 parts of anhydrous sodium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 1.7% sodium citrate.

### Example 2

Added to a mixture of soy protein curd and whey protein, wherein there are 70 parts of the protein curd containing 10% soy protein are 0.1 parts of anhydrous citric acid and 0.087 parts of anhydrous potassium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 2.0% potassium citrate.

### Example 3

Added to a mixture of soy protein curd and whey protein, wherein there are 70 parts of the protein curd containing 10% soy protein are 0.32 parts of anhydrous citric acid and 0.20 parts of anhydrous sodium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 5.244% sodium citrate.

### Example 4

Added to a mixture of soy protein curd and whey protein, wherein there are 70 parts of the protein curd containing 10% soy protein are 0.32 parts of anhydrous citric acid and 0.28 parts of anhydrous potassium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 6.2% potassium citrate.

### Example 5

Added to a mixture of soy protein curd and whey protein, wherein there are 70 parts of the protein curd containing 10% soy protein are 0.4 parts of anhydrous citric acid and 0.25 parts of anhydrous sodium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 6.45% sodium citrate.

### Example 6

Added to a mixture of soy protein curd and whey protein, wherein there are 70 parts of the protein curd containing 10% soy protein are 0.4 parts of anhydrous citric acid and 0.35 parts of anhydrous potassium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 7.56% potassium citrate.

### Example 7

Added to a mixture of soy protein curd and whey protein, wherein there are 70 parts of the protein curd containing 10% soy protein are 0.54 parts of anhydrous citric acid and 0.337 parts of anhydrous sodium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 8.52% sodium citrate.

### Example 8

Added to a mixture of soy protein curd and whey protein, wherein there are 70 parts of the protein curd containing 10% soy protein are 0.54 parts of anhydrous citric acid and 0.472 parts of anhydrous potassium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 9.94% potassium citrate.

### Example 9

Added to a mixture of soy protein curd and whey protein, wherein there are 70 parts of the protein curd containing 10% soy protein are 0.364 parts of 85% phosphoric acid and 0.38 parts of anhydrous sodium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 6.24% sodium citrate.

### Example 10

Added to a mixture of soy protein curd and whey protein, wherein there are 70 parts of the protein curd containing 10% soy protein are 0.364 parts of anhydrous citric acid and 0.532 parts of anhydrous potassium hydroxide. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 7.93% potassium citrate.

### Example 11

Added to a mixture of soy protein curd and whey protein, wherein there are 95 parts of the protein curd containing 10% soy protein are 0.30 parts of solid potassium phosphate, dibasic and 0.15 parts of solid sodium hexametaphosphate and 90.1 parts water. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 3.03% of potassium phosphate, dibasic and about 1.52% of sodium hexametaphosphate.

### Example 12

Added to a mixture of soy protein curd and whey protein, wherein there are 95 parts of the protein curd containing 10% soy protein are 0.75 parts of solid potassium citrate. The contents are stirred and heated to 85° C. and held at that temperature for 5 minutes. The contents are then subjected to a two stage homogenization at 2500/500 pounds per square inch. The contents are spray dried at an inlet temperature of 180° C. and an outlet temperature of 90° C. The product is a soy protein isolate containing about 7.35% of potassium citrate.

Examples 13 and 14 are the inventive soy protein isolates that contain between 1 and 10 wt. % of a metal salt wherein the metal of the metal salt is selected from the group consisting of sodium, potassium, and combinations thereof. Beverages are prepared in Examples 15-18 that use the soy protein isolates of Examples 13 and 14.

### Example 13

Added to a vessel are 17.96 kilograms of water and 150 grams of potassium citrate. The contents are stirred and heated up to about 60° C. and 1.89 kilograms of Supro XT219D are added. The temperature is increased to about 80° C. and the Supro XT219D slurry is permitted to hydrate for about 15 minutes. The slurry is then passed through a jet cooker at a pressure of 85 pounds per square inch. The steam heats the slurry in the jet cooker to a temperature of 200° C. After 8-10 seconds, progressive portions of the heated slurry are discharged into a receiver at below atmospheric pressure. The mineral fortified slurry is then spray dried to a moisture level of less than 5% by weight to give a soy protein isolate containing about 7.35 wt. % of potassium citrate.

### Example 14

Added to a vessel are 18.02 kilograms of water, 60 grams of dibasic potassium phosphate, and 30 grams of sodium hexametaphosphate. The contents are stirred and heated up to about 60° C. and 1.89 kilograms of Supro XT219D are added. The temperature is increased to about 80° C. and the Supro XT219D slurry is permitted to hydrate for about 15 minutes. The slurry is then passed through a jet cooker at a pressure of 85 pounds per square inch. The steam heats the slurry in the jet cooker to a temperature of 200° C. After 8-10 seconds, progressive portions of the heated slurry are discharged into a receiver at below atmospheric pressure. The mineral fortified slurry is then spray dried to a moisture level of less than 5% by weight to give a soy protein isolate containing about 4.55 wt. % of a combination of dibasic potassium phosphate and sodium hexametaphosphate.

### The Beverage

Formation of protein-containing sediment is a common problem for acidic beverages as well as neutral beverages that have been fortified with soy protein, due to the insolubility of the soy protein in the aqueous environment present in the drinks. Soy protein products used in beverages also often have a poor aftertaste. The isolated soy protein compositions of the present invention are suitable for use in beverages, while having less or no sedimentation and a reduction in astringent aftertaste commonly associated with isolated soy protein fortified beverages.

Because the isolated soy protein compositions of the present invention have improved solubility in beverages, they are ideally suited for use in acid and neutral, protein-containing drinks. Thus, in one embodiment, the protein-containing beverage composition is formulated using a soy protein isolate of the present invention. Preferably, the, protein-containing beverage composition comprises from about 0.5 wt. % to about 15 wt. % of the soy protein isolate, and more preferably from about 1.5 wt. % to about 10 wt. % of the soy protein isolate.

In preparing the protein-containing drinks, it is preferable to first hydrate the soy protein isolate to increase the solubility of the soy protein material in an aqueous solution. Methods for hydrating soy protein material are known in the art. Briefly, the soy protein product may be hydrated by dispersing the soy protein isolate or concentrate in an aqueous solution, preferably water or a pH adjusted aqueous alkali, having a pH significantly above or below the isoelectric point of the protein, preferably a pH of greater than 5.5 or less than 3.0, so the protein does not precipitate out from the solution. The amount of protein hydrated in the aqueous solution is preferably from about 0.6% to about 16% (by weight of the final protein-containing drink), and the amount of aqueous solution in which the protein is hydrated is preferably at least 4 times the amount of protein material by weight. Preferably, the aqueous solution in which the protein material is hydrated is from 65% to 90% by weight of the final protein-containing drink.

The aqueous solution in which the soy protein product is hydrated is preferably heated above ambient temperature prior to, or upon, addition of the soy protein product to the hydrating solution to facilitate hydration of the soy protein product. Preferably the aqueous hydrating solution is heated to a temperature of from about 110° F. to about 170° F. (about 43° C. to about 77° C.) to aid in the hydration of the protein material therein, and is preferably maintained at this temperature for about 5 to about 60 minutes, preferably for about 10 minutes. The temperature of the hydrating solution may be further adjusted, if desired, to speed the hydration of the protein material. Preferably the temperature of the hydrating solution is adjusted to a temperature of from about 150° F. to about 180° F. (about 65° C. to about 82° C.).

After hydration of the isolated soy protein composition, non-acidic flavoring agents, defoamers, coloring agents, nutrients, and sweeteners may be added to the aqueous hydrating solution. Flavoring agents include commercially available natural and artificial flavors, including concentrated fruit or vegetable juices. Coloring agents may be commercially available natural and artificial colorants. Preferred sweeteners are carbohydrates such as sucrose and fructose, and include high fructose corn syrup. Nutrients such as vitamins and minerals may also be added.

After addition of the non-acidic flavoring agents, coloring agents, defoamers, nutrients, and sweeteners to the aqueous hydrating solution, the hydrating solution is optionally mixed until the added components are thoroughly distributed in the hydrating solution. If the temperature of the hydrating solution has not already been adjusted, the temperature of the hydrating solution may be increased when mixing the added components to ensure that the ingredients in the hydrating solution are optimally mixed. Preferably the temperature of the hydrating solution is raised to about 150° F. to about 180° F. (about 65° C. to about 82° C.).

After the isolated soy protein composition is hydrated in the hydrating solution and any desired flavoring agents, coloring agents, defoamers, sweeteners, and nutrients are mixed in the hydrating solution, the hydrating solution is adjusted, if needed, to the desired pH of the final protein-containing drink. The protein-containing drinks of the present invention typically have a pH of from about 2.5 to about 8.0, preferably from about 3.0 to about 7.5, and more preferably from about 6.0 to about 7.0. If needed, the hydrating solution may be acidified to the desired pH by adding an acidulent such as an edible acid (e.g. lactic acid, citric acid, phosphoric acid) to the hydrating solution, by mixing the hydrating solution with an acidic liquid such as a fruit or a vegetable juice, by mixing the hydrating solution with an acidic fruit or vegetable juice concentrate, or by combinations of such methods. Alternately, the hydrating solution may be made more basic, if needed, by adding a base, preferably a dilute alkaline solution such as an aqueous sodium or potassium hydroxide solution, or by adding sufficient quantities of a juice or juice concentrate to raise the pH of the hydrating solution to the desired pH of the acidic, protein-containing drink. Most preferably, the hydrating solution containing the isolated soy protein composition and other ingredients is acidified to a pH other than the isoelectric point of the protein material to avoid maximum insolubility of the protein in the acidified solution.

In one embodiment of the invention, the hydrating solution containing the hydrated isolated soy protein composition and other ingredients is mixed with a juice or a juice concentrate to provide a liquid drink. Preferred fruit and vegetable juices and juice concentrates include apple juice, grape juice, orange juice, carrot juice, lemon juice, lime juice, grapefruit juice, pineapple juice, cranberry juice, peach juice, pear juice, celery juice, cherry juice, tomato juice, passion fruit juice, mango juice, blends thereof, and their concentrates. If the desired pH of the aqueous liquid drink is lower than the pH provided by mixing the juice and/or juice concentrate with the hydrating solution, the pH may be further lowered by adding an edible acidulent to the mixture. Preferred edible acidulents include citric acid, lactic acid, and phosphoric acid.

In another embodiment of the invention the hydrating solution containing the hydrated isolated soy protein composition and other ingredients is acidified by adding a sufficient amount of one or more edible acidulents and, if desired, additional flavoring, coloring agents, nutrients, and sweeteners, to the hydrating solution to adjust the pH of the hydrating solution to the desired acid pH. As noted above, preferred edible acidulents include citric acid, lactic acid, phosphoric acid, and ascorbic acid, among others.

In most cases, the protein-containing drink will be pasteurized or sterilized to eliminate any microbial contamination of the suspension and enable storage stability. The protein-containing drink is preferably pasteurized with conventional pasteurization equipment at a temperature of from about 175° F. to about 215° F. (about 80° C. to about 101.7° C.) for 0.5 to 3 minutes.

Because of the good solubility of the isolated soy protein compositions used in the preparation of the protein-containing drinks, there is generally no need to homogenize the protein-containing drinks after the ingredients have been added. However, if the protein-containing drink is not completely mixed or similar in particle size, it may optionally be homogenized before or after pasteurization. Homogenization may be done by any conventional technique, for example, by high pressure treatment at 1000 to 5000 pounds per square inch utilizing a valve-type Rannie or Gaulin homogenizer. Optimally the homogenization is done in two stages with the first stage at 2500 pounds per square inch and the second stage at 500 pounds per square inch.

Many prior processes of preparing protein-containing drinks have used protein stabilizing agents to attempt to improve the solubility and stability of soy proteins present in the protein-containing drinks. In general, stabilizing agents interact with the surfaces of the soy proteins (and other components of the acidic, protein-containing drinks) and stabilize the electrostatic interaction between proteins. This in turn reduces the formation of large protein aggregates, which have reduced solubility and stability in acidic environments, and tend to precipitate out of solution. Stabilization agents are typically required in ready to drink beverages and typically are optional in dry blended beverages. Commonly used stabilizing agents include, for example, pectin, polysaccharide hydrolysates comprising dextrin, agar, can-ageenan, tamarind seed polysaccharides, angelica gum, karaya gum, xanthan gum, sodium alginate, tragacanth gum, guar gum, locust bean gum, pullulan, gellan gum, gum arabic, carboxymethylcellulose, and propylene glycol alginate ester.

Since the isolated soy protein compositions used to prepare the protein-containing drinks have good solubility and suspendability, there is generally little to no sediment in the protein-containing drinks of the present invention. The sedimentation rate may be determined by using, for example, the following method: Hot-fill a sample of the protein-containing drink into a 250 ml Nalgene narrow moth square bottle up to 2 mm from the top. Tightly close the bottle and invert the bottle for about 3 minutes to sterilize the lid and top of the bottle. Place the filled bottle into an ice bath for about 30 minutes to cool the protein-containing drink to room temperature. Put the bottle into storage at room temperature for 30 days. After 30 days, measure from the benchtop to top of the sediment and deduct 2 mm from the reading for the height of the bottom of the bottle to obtain the height of the sediment. Measure the total volume height. Divide the height of the sediment by the total volume height and multiply the resulting number by 100 to obtain the sedimentation rate. Optionally, this procedure may be repeated and the average of the readings calculated. The sedimentation rate of the protein-containing drink after 30 days of ambient storage will preferably be less than about 1.0% by volume, preferably less than about 0.5% by volume, and optimally is less than about 0.1% by volume, as measured by this test.

The protein-containing drinks containing the isolated soy protein composition of the present invention can further have a shorter shake back time as compared to, protein-containing drinks made using conventional soy protein products.

### EXAMPLES

The following examples are simply intended to further illustrate and explain the present invention. The invention, therefore, should not be limited to any of the details in these examples.

### Example 15

About 9.9 parts of the dry soy protein isolate from Example 11, 90.1 parts milk, and 10 parts chocolate flavoring are combined and permitted to hydrate. The contents are stirred to prepare a beverage.

### Example 16

A 6.5 g protein per 8 oz serving fortified ready to drink beverage is made using the dry soy protein isolate from Example 11.

Added to a vessel is 150 g sodium citrate in 5000 g deionized water. After the sodium citrate has dissolved, 153 g of the product from Example 11 is added. The contents are heated to 82° C. and held for 8 minutes to hydrate the protein material. In a separate vessel, a stabilizing agent is prepared by dry mixing 150 g pectin and 300 g sucrose, which is then added to the hydrated protein vessel to hydrate the pectin. A flavoring material as a solution of 400 g sucrose, 164 g apple juice concentrate and 350 citric acid is added to the protein containing vessel and the blend is pasteurized at 90° C. for 60 seconds, followed by homogenization in two stages, a high pressure stage of 2500 pounds per square inch and a low pressure stage of 500 pounds per square inch. The pH is 3.86. Bottles are hot filled, inverted for 2 minutes and then placed in ice water to bring the temperature of the contents to about room temperature.

### Example 17

Supro XT219D, available from Solae, LLC, St Louis, Mo. is used as a control for a dry blended beverage. Added to a vessel are 6.3 grams of Supro XT219D and 30.625 grams of a dry blended beverage base from Ross Labs, Columbus, Ohio. The dry blended beverage base contains stabilizers, flavorants, sweeteners, and vitamins. The contents of the vessel are dry blended. The contents are then combined with 8 ounces of cold water and blended for 15 seconds at low speed using an Osterizer blender.

### Example 18

Added to a vessel are 6.62 grams of the dry soy protein isolate of Example 14 and 30.62 grams of the dry blended beverage base from Ross Labs. The contents of the vessel are dry blended. The contents are then combined with 8 ounces of cold water and blended for 15 seconds at low speed using an Osterizer blender.

The acceptability of the beverage compositions of the present invention, includes the organoleptic acceptability, which can be measured, for example by determining the value on a nine-point hedonic scale. A composition is considered, herein, to be organoleptically acceptable if a combination of the appearance, flavor, and mouthfeel of the composition score at least about four or greater on a nine-point hedonic scale.

When determining the overall acceptance rating of the beverage product, the beverage product was evaluated by a panel of 9 panelists (males and females, ages 35-54). The beverage of Example 17 is used as a control. The test beverage products are evaluated using blind product paired test. A nine point hedonic scale is used to judge the overall acceptability of the beverage products. Such scale and methodology can be found on pages 101-103 and 213 of Sensory Evaluation Techniques, 2^{nd} Edition by Morten Meilgaard et al., CRC Press, 1991.

Standard testing procedures for sensory evaluation are known in the alt including, in particular, a 9-point hedonic scale as described below (see Stone and Sidel in Sensory Evaluation Practices, Academic Press, Orlando, 1985, pp 58-86, 227-252). Compositions scoring above neutral on a 9-point hedonic scale, i.e. 5.0 or greater, are considered to be acceptable with respect to those attributes.

Experimental samples were evaluated for Flavor and Mouthfeel on a standard nine-point hedonic scale. The scale is as follows:
Score/rating Std. Hedonic Scale
   9 Like extremely
   8 Like very much
   7 Like moderately
   6 Like slightly
   5 Neither like nor dislike
   4 Dislike slightly
   3 Dislike moderately
   2 Dislike very much
   1 Dislike extremely

The inventive beverage product of Example 18 is 2.6 hedonic units higher than that of Example 17.

While the invention has been explained in relation to its preferred embodiments, it is to be understood that various modifications thereof will become apparent to those skilled in the art upon reading the description. Therefore, it is to be understood that the invention disclosed herein is intended to cover such modifications as fall within the scope of the appended claims.

What is claimed is:

1. A soy protein isolate composition, comprising; a soy protein isolate that contains from about 1.0 wt. % up to about 10 wt. % of a metal salt, wherein the metal of the metal salt is selected from the group consisting of sodium, potassium, and combinations thereof.

2. The composition of claim 1, wherein the metal salt is selected from the group consisting of a metal citrate, a metal phosphate, a metal hexametaphosphate, and combinations thereof.

3. The composition of claim 2, wherein the metal citrate is selected from the group consisting of sodium citrate, potassium citrate, and combinations thereof.

4. The composition of claim 2, wherein the metal phosphate is selected from the group consisting of phosphates of sodium, potassium, and combinations thereof.

5. The composition of claim 4, wherein the phosphates are tribasic phosphates, dibasic phosphates, monobasic phosphates, and mixtures thereof.

6. The composition of claim 2, wherein the metal hexametaphosphate is selected from the group consisting of sodium hexametaphosphate, potassium hexametaphosphate, and combinations thereof.

7. The composition of claim 1, wherein the metal salt is present at from about 3 wt. % up to about 8 wt. %.

8. A process for preparing an isolated soy protein composition, comprising;
preparing an aqueous extract from a protein containing material,
adjusting the pH of the aqueous extract to a value of from about 4 to about 5 to precipitate the protein material,
separating the precipitated protein material and forming a suspension of the precipitated protein material in water,
adding a metal salt to the separated precipitated protein material suspension, wherein the metal of the metal salt is selected from the group consisting of sodium, potassium, and combinations thereof
homogenizing the precipitated protein material suspension containing the metal salt,
removing the protein material from the suspension, and
spray drying the protein material to obtain an isolated soy protein composition containing from about 1 wt. % up to about 10 wt. % of the metal salt.

9. A beverage composition, comprising;
a hydrated isolated soy protein composition, wherein the soy protein isolate contains from about 1.0 wt. % up to about 10 wt. % of a metal salt, wherein the metal of the metal salt is selected from the group consisting of sodium, potassium, and combinations thereof;
a liquid selected from the group consisting of a juice or a juice concentrate and a milk product; and
at least one selected from the group consisting of flavoring agents, coloring agents, defoamers, sweeteners, nutrients, and mixtures thereof.

10. The composition of claim 9, wherein the metal salt is selected from the group consisting of a metal citrate, a metal phosphate, a metal hexametaphosphate, and combinations thereof.

11. The composition of claim 10, wherein the metal citrate is selected from the group consisting of sodium citrate, potassium citrate, and combinations thereof.

12. The composition of claim 10, wherein the metal phosphate is selected from the group consisting of phosphates of sodium, potassium, and combinations thereof.

13. The composition of claim 12, wherein the phosphates are tribasic phosphates, dibasic phosphates, monobasic phosphates, and mixtures thereof.

14. The composition of claim 10, wherein the metal hexametaphosphate is selected from the group consisting of sodium hexametaphosphate, potassium hexametaphosphate, and combinations thereof.

15. The composition of claim 10, wherein the metal salt is present at from about 3 wt. % up to about 8 wt. %.
